(19) United States
(12) Patent Application Publication
(10) Pub. No.: US 2021/0142916 A1
(43) Pub. Date: May 13, 2021

(54) Document Management System and Method

(71) Applicant: Med-Legal Technologies, LLC, San Dimas, CA (US)

(72) Inventors: Gregory Scott Webber, Alpharetta, GA (US); Todd Morey Breyman, Johns Creek, GA (US); Joseph James Tuthill, Chino, CA (US)

(21) Appl. No.: 17/094,991

(22) Filed: Nov. 11, 2020

Related U.S. Application Data
(60) Provisional application No. 62/934,238, filed on Nov. 12, 2019.

(51) Int. Cl. G16H 50/70; G16H 10/60; G06F 16/93; G06F 16/9035; G06F 16/9038; H04L 9/06

(52) U.S. Cl. CPC G16H 50/70; G16H 10/60; G06F 16/93; H04L 2209/38; G06F 16/9038; H04L 9/0643; G06F 16/9035

(57) ABSTRACT

The present disclosure relates to a document management system and a computer-implemented method. Embodiments may include receiving, using a processor, information relating to a case being adjudicated at an electronic storage system. Embodiments may also include identifying, using a categorization process, one or more evidentiary elements relevant to the case being adjudicated and displaying information at a graphical user interface, the information including a view of a history, progression and a status of a medical condition associated with the case being adjudicated. Embodiments may further include generating a report or analysis based upon, at least in part, the information.

### CROSS-REFERENCE TO RELATED APPLICATIONS

The subject application claims the benefit of U.S. Provisional Patent Application with Ser. No. 62/934,238, filed Nov. 12, 2019, entitled Document Management System and Method, the entire content of which are herein incorporated by reference.

### BACKGROUND

Many legal proceedings require some form of discovery where parties make particular information or documents available for review. Electronic discovery has introduced many conveniences associated with accessing electronic information but has also introduced data security and document management concerns.

### BRIEF SUMMARY OF DISCLOSURE

In one example implementation, a method, performed by one or more computing devices, may include but is not limited to receiving, using a processor, information relating to a case being adjudicated at an electronic storage system. The method may further include identifying, using a categorization process, on or more evidentiary elements relevant to the case being adjudicated and displaying information at a graphical user interface, the information including a view of a history, progression and a status of a medical condition associated with the case being adjudicated. The method may also include generating a report or analysis based upon, at least in part, the information.

One or more of the following example features may be included. The method may include encoding case record information in a Record Annotation Markup Language “RAML” file. The method may also include generating a case record excerpt summary including at least one of dates of service, service provider, summary of pertinent facts, or a list of page numbers. The method may further include graphically displaying a chronological view of the case including at least one of case facts, dates, item description, case record title or case record type. The method may also include generating a real-time updated view of all evidence associated with the case being adjudicated. The method may further include rating one or more records with respect to a static or a dynamic list of factors. The method may also include displaying, at a graphical user interface, a complete medical history portal including a plurality of graphical user interfaces configured to allow a user to search or order one or more records. The method may include displaying, at a graphical user interface, a record availability dashboard. The method may further include selecting, via a graphical user interface, one or more documents to include in a bundle of documents within an electronic storage system, defining, via the user interface, accessibility constraints for the one or more documents, and electronically sharing at least one document of the bundle of documents with another user based upon, at least in part, the defined accessibility constraints. The method may also include automatically adding one or more case documents to the bundle of documents. The method may also include creating, editing, or deleting a bundle of documents. The method may further include enabling a bundle updating service configured to identify one or more case records to update one or more live bundles with all available records. The method may also include generating a subset of disputes of the case being adjudicated, generating one or more active arguments related to the subset of disputes, and/or automatically generating one or more packaged settlement documents. The method may further include authenticating the information using one or more of blockchain, digital rights management, digital signatures, or encryption processes. The blockchain process may include a trust ledger data store. The method may further include displaying, at a graphical user interface, a current and verified capability to track, view, personally utilize, notate, add private documentation, share, or report on all evidentiary discovery elements that are active and in use for a case by a case party or authorized participant for an adjudicated case. The method may also include displaying, at a graphical user interface, a current, immediate, and verified view to active and authorized case parties for the case being adjudicated. The method may also include performing updating operations regarding one or more data updates to an Electronic Adjudication Management System “EAMS” using one or more synchronization engines.

The details of one or more example implementations are set forth in the accompanying drawings and the description below. Other possible example features and/or possible example advantages will become apparent from the description, the drawings, and the claims. Some implementations may not have those possible example features and/or possible example advantages, and such possible example features and/or possible example advantages may not necessarily be required of some implementations.

### BRIEF DESCRIPTION OF THE DRAWINGS

FIG. 1 is an example diagrammatic view of a document management process coupled to an example distributed computing network according to one or more example implementations of the disclosure;

FIG. 2 is an example flowchart of a document management process according to one or more example implementations of the disclosure;

FIGS. 3-7 are example diagrammatic views of various user interfaces of the document management process according to one or more example implementations of the disclosure;

FIGS. 8-9 are example flowcharts of the document management process according to one or more example implementations of the disclosure;

FIGS. 10-11 are example diagrammatic views of various user interfaces of the document management process according to one or more example implementations of the disclosure;

FIGS. 12-13 are example flowcharts of the document management process according to one or more example implementations of the disclosure;

FIG. 14 is an example diagrammatic view of a user interface of the document management process according to one or more example implementations of the disclosure;

FIG. 15 is an example flowchart of the document management process according to one or more example implementations of the disclosure;

FIG. 16 is an example diagrammatic view of a user interface of the document management process according to one or more example implementations of the disclosure;

FIG. 17 is an example flowchart of the document management process according to one or more example implementations of the disclosure;

FIGS. 18-25 are example diagrammatic views of various user interfaces of the document management process according to one or more example implementations of the disclosure;

FIGS. 26-31 are example flowcharts of the document management process according to one or more example implementations of the disclosure; and

FIG. 32 is an example diagrammatic view of a computer of FIG. 1 according to one or more example implementations of the disclosure.

Like reference symbols in the various drawings indicate like elements.

### DETAILED DESCRIPTION

### System Overview:

In some implementations, the present disclosure may be embodied as a method, system, or computer program product. Accordingly, in some implementations, the present disclosure may take the form of an entirely hardware implementation, an entirely software implementation (including firmware, resident software, micro-code, etc.) or an implementation combining software and hardware aspects that may all generally be referred to herein as a “circuit,” “module” or “system.” Furthermore, in some implementations, the present disclosure may take the form of a computer program product on a computer-usable storage medium having computer-usable program code embodied in the medium.

In some implementations, any suitable computer usable or computer readable medium (or media) may be utilized. The computer readable medium may be a computer readable signal medium or a computer readable storage medium. The computer-usable, or computer-readable, storage medium (including a storage device associated with a computing device or client electronic device) may be, for example, but is not limited to, an electronic, magnetic, optical, electromagnetic, infrared, or semiconductor system, apparatus, device, or any suitable combination of the foregoing. More specific examples (a non-exhaustive list) of the computer-readable medium may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a digital versatile disk (DVD), a static random access memory (SRAM), a memory stick, a floppy disk, a mechanically encoded device such as punch-cards or raised structures in a groove having instructions recorded thereon, a media such as those supporting the internet or an intranet, or a magnetic storage device. Note that the computer-usable or computer-readable medium could even be a suitable medium upon which the program is stored, scanned, compiled, interpreted, or otherwise processed in a suitable manner, if necessary, and then stored in a computer memory. In the context of the present disclosure, a computer-usable or computer-readable, storage medium may be any tangible medium that can contain or store a program for use by or in connection with the instruction execution system, apparatus, or device.

In some implementations, a computer readable signal medium may include a propagated data signal with computer readable program code embodied therein, for example, in baseband or as part of a carrier wave. In some implementations, such a propagated signal may take any of a variety of forms, including, but not limited to, electro-magnetic, optical, or any suitable combination thereof. In some implementations, the computer readable program code may be transmitted using any appropriate medium, including but not limited to the internet, wireline, optical fiber cable, RF, etc. In some implementations, a computer readable signal medium may be any computer readable medium that is not a computer readable storage medium and that can communicate, propagate, or transport a program for use by or in connection with an instruction execution system, apparatus, or device.

In some implementations, computer program code for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java®, Smalltalk, C++ or the like. Java® and all Java-based trademarks and logos are trademarks or registered trademarks of Oracle and/or its affiliates. However, the computer program code for carrying out operations of the present disclosure may also be written in conventional procedural programming languages, such as the “C” programming language, PASCAL, or similar programming languages, as well as in scripting languages such as Javascript, PERL, or Python. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through a local area network (LAN) or a wide area network (WAN), or the connection may be made to an external computer (for example, through the internet using an Internet Service Provider). In some implementations, electronic circuitry including, for example, programmable logic circuitry, field-programmable gate arrays (FPGAs) or other hardware accelerators, micro-controller units (MCUs), or programmable logic arrays (PLAs) may execute the computer readable program instructions/code by utilizing state information of the computer readable program instructions to personalize the electronic circuitry, in order to perform aspects of the present disclosure.

In some implementations, the flowchart and block diagrams in the figures illustrate the architecture, functionality, and operation of possible implementations of apparatus (systems), methods and computer program products according to various implementations of the present disclosure. Each block in the flowchart and/or block diagrams, and combinations of blocks in the flowchart and/or block diagrams, may represent a module, segment, or portion of code, which comprises one or more executable computer program instructions for implementing the specified logical function(s)/act(s). These computer program instructions may be provided to a processor of a general purpose computer, special purpose computer, or other programmable data processing apparatus to produce a machine, such that the computer program instructions, which may execute via the processor of the computer or other programmable data processing apparatus, create the ability to implement one or more of the functions/acts specified in the flowchart and/or block diagram block or blocks or combinations thereof. It should be noted that, in some implementations, the functions noted in the block(s) may occur out of the order noted in the figures (or combined or omitted). For example, two blocks shown in succession may, in fact, be executed substantially concurrently, or the blocks may sometimes be executed in the reverse order, depending upon the functionality involved.

In some implementations, these computer program instructions may also be stored in a computer-readable memory that can direct a computer or other programmable data processing apparatus to function in a particular manner, such that the instructions stored in the computer-readable memory produce an article of manufacture including instruction means which implement the function/act specified in the flowchart and/or block diagram block or blocks or combinations thereof.

In some implementations, the computer program instructions may also be loaded onto a computer or other programmable data processing apparatus to cause a series of operational steps to be performed (not necessarily in a particular order) on the computer or other programmable apparatus to produce a computer implemented process such that the instructions which execute on the computer or other programmable apparatus provide steps for implementing the functions/acts (not necessarily in a particular order) specified in the flowchart and/or block diagram block or blocks or combinations thereof.

Referring now to the example implementation of FIG. 1, there is shown document management process 10 that may reside on and may be executed by a computer (e.g., computer 12), which may be connected to a network (e.g., network 14) (e.g., the internet or a local area network). Examples of computer 12 (and/or one or more of the client electronic devices noted below) may include, but are not limited to, a storage system (e.g., a Network Attached Storage (NAS) system, a Storage Area Network (SAN)), a personal computer(s), a laptop computer(s), mobile computing device(s), a server computer, a series of server computers, a mainframe computer(s), or a computing cloud(s). As is known in the art, a SAN may include one or more of the client electronic devices, including a RAID device and a NAS system. In some implementations, each of the aforementioned may be generally described as a computing device. In certain implementations, a computing device may be a physical or virtual device. In many implementations, a computing device may be any device capable of performing operations, such as a dedicated processor, a portion of a processor, a virtual processor, a portion of a virtual processor, portion of a virtual device, or a virtual device. In some implementations, a processor may be a physical processor or a virtual processor. In some implementations, a virtual processor may correspond to one or more parts of one or more physical processors. In some implementations, the instructions/logic may be distributed and executed across one or more processors, virtual or physical, to execute the instructions/logic. Computer 12 may execute an operating system, for example, but not limited to, Microsoft® Windows®; Mac® OS X®; Red Hat® Linux®, Windows® Mobile, Chrome OS, Blackberry OS, Fire OS, or a custom operating system. (Microsoft and Windows are registered trademarks of Microsoft Corporation in the United States, other countries or both; Mac and OS X are registered trademarks of Apple Inc. in the United States, other countries or both; Red Hat is a registered trademark of Red Hat Corporation in the United States, other countries or both; and Linux is a registered trademark of Linus Torvalds in the United States, other countries or both).

In some implementations, as will be discussed below in greater detail, a document management process, such as document management process 10 of FIG. 1, may include receiving, via a computing device, transaction data associated with a plurality of distinct point-of-sale systems. The transaction data from the plurality of distinct point-of-sale systems may be mapped to a standardized hierarchy of components. The standardized hierarchy of components, including aggregated mapped transaction data, may be shared.

In some implementations, the instruction sets and subroutines of document management process 10, which may be stored on storage device, such as storage device 16, coupled to computer 12, may be executed by one or more processors and one or more memory architectures included within computer 12. In some implementations, storage device 16 may include but is not limited to: a hard disk drive; all forms of flash memory storage devices; a tape drive; an optical drive; a RAID array (or other array); a random access memory (RAM); a read-only memory (ROM); or combination thereof. In some implementations, storage device 16 may be organized as an extent, an extent pool, a RAID extent (e.g., an example 4D+1P R5, where the RAID extent may include, e.g., five storage device extents that may be allocated from, e.g., five different storage devices), a mapped RAID (e.g., a collection of RAID extents), or combination thereof.

In some implementations, network 14 may be connected to one or more secondary networks (e.g., network 18), examples of which may include but are not limited to: a local area network; a wide area network; or an intranet, for example.

In some implementations, computer 12 may include a data store, such as a database (e.g., relational database, object-oriented database, triplestore database, etc.) and may be located within any suitable memory location, such as storage device 16 coupled to computer 12. In some implementations, data, metadata, information, etc. described throughout the present disclosure may be stored in the data store. In some implementations, computer 12 may utilize any known database management system such as, but not limited to, DB2, in order to provide multi-user access to one or more databases, such as the above noted relational database. In some implementations, the data store may also be a custom database, such as, for example, a flat file database or an XML database. In some implementations, any other form(s) of a data storage structure and/or organization may also be used. In some implementations, document management process 10 may be a component of the data store, a standalone application that interfaces with the above noted data store and/or an applet/application that is accessed via client applications 22, 24, 26, 28. In some implementations, the above noted data store may be, in whole or in part, distributed in a cloud computing topology. In this way, computer 12 and storage device 16 may refer to multiple devices, which may also be distributed throughout the network.

In some implementations, computer 12 may execute a client application (e.g., client application 20), examples of which may include, but are not limited to, e.g., point-of-sale applications/systems, performance reporting applications/systems, inventory applications/systems, delivery applications/systems, analytics applications/systems, ordering applications/systems, etc. In some implementations, document management process 10 and/or client application 20 may be accessed via one or more of client-side applications 22, 24, 26, 28. In some implementations, document management process 10 may be a standalone application, or may be an applet/application/script/extension that may interact with and/or be executed within client application 20, a component of client application 20, and/or one or more of client-side applications 22, 24, 26, 28. In some implementations, client application 20 may be a standalone application, or may be an applet/application/script/extension that may interact with and/or be executed within document management process 10, a component of document management process 10, and/or one or more of client-side applications 22, 24, 26, 28. In some implementations, one or more of client-side applications 22, 24, 26, 28 may be a standalone application, or may be an applet/application/script/extension that may interact with and/or be executed within and/or be a component of document management process 10 and/or client application 20. Examples of client-side applications 22, 24, 26, 28 may include, but are not limited to, e.g., point-of-sale applications (e.g., executed by a point-of-sale terminal), performance reporting applications/systems, inventory applications/systems, delivery applications/systems, analytics applications/systems, ordering applications/systems, a standard and/or mobile web browser, an email application (e.g., an email client application), a textual and/or a graphical user interface, a customized web browser, a plugin, an Application Programming Interface (API), or a custom application. The instruction sets and subroutines of client-side applications 22, 24, 26, 28, which may be stored on storage devices 30, 32, 34, 36, coupled to client electronic devices 38, 40, 42, 44, may be executed by one or more processors and one or more memory architectures incorporated into client electronic devices 38, 40, 42, 44.

In some implementations, one or more of storage devices 30, 32, 34, 36, may include but are not limited to: hard disk drives; flash drives, tape drives; optical drives; RAID arrays; random access memories (RAM); and read-only memories (ROM). Examples of client electronic devices 38, 40, 42, 44 (and/or computer 12) may include, but are not limited to, a personal computer (e.g., client electronic device 38), a laptop computer (e.g., client electronic device 40), a smart/data-enabled, cellular phone (e.g., client electronic device 42), a notebook computer (e.g., client electronic device 44), a tablet, a point-of-sale terminal (e.g., computing device used to process and record transactions), a server, a television, a smart television, a media (e.g., video, photo, etc.) capturing device, and a dedicated network device. In some implementations, each of client electronic devices 38, 40, 42, 44 may each execute a point-of-sale application/system for recording transactions. Accordingly and in some implementations, client devices 38, 40, 42, 44 may be considered point-of-sale terminals. Client electronic devices 38, 40, 42, 44 may each execute an operating system, examples of which may include but are not limited to, Android™, Apple® iOS®, Mac® OS X®; Red Hat® Linux®, Windows® Mobile, Chrome OS, Blackberry OS, Fire OS, or a custom operating system.

In some implementations, one or more of client-side applications 22, 24, 26, 28 may be configured to effectuate some or all of the functionality of document management process 10 (and vice versa). Accordingly, in some implementations, document management process 10 may be a purely server-side application, a purely client-side application, or a hybrid server-side/client-side application that is cooperatively executed by one or more of client-side applications 22, 24, 26, 28 and/or document management process 10.

In some implementations, one or more of client-side applications 22, 24, 26, 28 may be configured to effectuate some or all of the functionality of client application 20 (and vice versa). Accordingly, in some implementations, client application 20 may be a purely server-side application, a purely client-side application, or a hybrid server-side/client-side application that is cooperatively executed by one or more of client-side applications 22, 24, 26, 28 and/or client application 20. As one or more of client-side applications 22, 24, 26, 28, document management process 10, and client application 20, taken singly or in any combination, may effectuate some or all of the same functionality, any description of effectuating such functionality via one or more of client-side applications 22, 24, 26, 28, document management process 10, client application 20, or combination thereof, and any described interaction(s) between one or more of client-side applications 22, 24, 26, 28, document management process 10, client application 20, or combination thereof to effectuate such functionality, should be taken as an example only and not to limit the scope of the disclosure.

In some implementations, one or more of users 46, 48, 50, 52 may access computer 12 and document management process 10 (e.g., using one or more of client electronic devices 38, 40, 42, 44) directly through network 14 or through secondary network 18. Further, computer 12 may be connected to network 14 through secondary network 18, as illustrated with phantom link line 54. Document management process 10 may include one or more user interfaces, such as browsers and textual or graphical user interfaces, through which users 46, 48, 50, 52 may access document management process 10.

In some implementations, the various client electronic devices may be directly or indirectly coupled to network 14 (or network 18). For example, client electronic device 38 is shown directly coupled to network 14 via a hardwired network connection. Further, client electronic device 44 is shown directly coupled to network 18 via a hardwired network connection. Client electronic device 40 is shown wirelessly coupled to network 14 via wireless communication channel 56 established between client electronic device 40 and wireless access point (i.e., WAP) 58, which is shown directly coupled to network 14. WAP 58 may be, for example, an IEEE 802.11a, 802.11b, 802.11g, 802.11n, 802.11ac, Wi-Fi®, RFID, and/or Bluetooth™ (including Bluetooth™ Low Energy) device that is capable of establishing wireless communication channel 56 between client electronic device 40 and WAP 58. Client electronic device 42 is shown wirelessly coupled to network 14 via wireless communication channel 60 established between client electronic device 42 and cellular network/bridge 62, which is shown by example directly coupled to network 14.

In some implementations, some or all of the IEEE 802.11x specifications may use Ethernet protocol and carrier sense multiple access with collision avoidance (i.e., CSMA/CA) for path sharing. The various 802.11x specifications may use phase-shift keying (i.e., PSK) modulation or complementary code keying (i.e., CCK) modulation, for example. Bluetooth™ (including Bluetooth™ Low Energy) is a telecommunications industry specification that allows, e.g., mobile phones, computers, smart phones, and other electronic devices to be interconnected using a short-range wireless connection. Other forms of interconnection (e.g., Near Field Communication (NFC)) may also be used.

In some implementations, various I/O requests (e.g., I/O request 15) may be sent from, e.g., client-side applications 22, 24, 26, 28 to, e.g., computer 12. Examples of I/O request 15 may include but are not limited to, data write requests (e.g., a request that content be written to computer 12) and data read requests (e.g., a request that content be read from computer 12).

### The Document Management Process:

As discussed above and referring also at least to the example implementations of FIGS. 2-32, document management process 10 may include but is not limited to receiving (200), using a processor, information relating to a case being adjudicated at an electronic storage system. The method may further include identifying (202), using a categorization process, on or more evidentiary elements relevant to the case being adjudicated and displaying (204) information at a graphical user interface, the information including a view of a history, progression and a status of a medical condition associated with the case being adjudicated. The method may also include generating (206) a report or analysis based upon, at least in part, the information.

### Active Bundling

In some implementations, document management process 10 may provide a unique capability to combine, package, and share documents (e.g., discovery/evidence) in turn making them usable and visible to other case parties and/or authorized participants in a case. Using any available document, then identified on a dashboard, the user can make appropriate packages and/or selections of the documents; either for internal reference and use—or ultimately for access/reference/use externally (other case party or participant). In some implementations, document management process 10 may generally create bundles of documents. A bundle may generally include collections of related documents created for specific purposes (e.g. highlighting case arguments, sharing documents for medical review, etc.). In the case of external bundles, bundles can also be assigned a ‘specialized’ form, fit for a particular purpose for (selected, UR, IMR, QME, AME) medical-legal evaluations (and in some cases directly connected to authorized providers/vendors). Additionally, bundles may take on ‘dynamic’ form, such that they may be automatically supplemented/updated with changes, updates, or additions as then specified. Such bundles may be securely shared and tracked.

In some implementations, via document management process 10, discovered information (e.g., evidence) becomes sharable with others; all through a proprietary and secure transmission. In this manner, the distribution of, visibility of, and use of required documentary evidence and information may be enhanced. Further, given the ease in securely sharing existing facts, information, and evidence (as maintained by document management process 10), the potential (and expense) of duplication may be reduced—saving the ecosystem substantial effort and expense. Finally, with dynamic bundles, updated information is easily shared and available in full context with the original packet of evidence.

In some implementations, document management process 10 may generally provide an internet-based electronic document management system for parties to conduct discovery related to and collaborate with others on those cases (e.g., in a workers compensation case). As will be discussed in greater detail below, various applications and services (e.g., Med-Legal) may grant authorized employees of its customers to access document management process 10 and to perform work in it based upon 1) their ability to authenticate themselves with document management process 10 by presenting their uniquely identifying user login credentials to the system upon entry, 2) the privileges that the user has been assigned to the system in another application (e.g., Med-Legal's CRM system), and/or 3) whether the customer account the user is associated with is a valid party to a case managed by document management process 10. In some implementations, document management process 10 may provide a collection of computer screens or user interfaces that provide the facilities to allow a user to filter, search, view, and manage functionality related to cases that they are a party to or a participant in.

In some implementations, document management process 10 may combine, package, and share documents (e.g., discovery/evidence) in turn making them usable and visible to other case parties and/or authorized participants in a case. A case party may generally include the applicant, defendant, legal representatives (e.g. applicant and defense attorneys), and/or claims administrators associated with a case (e.g., a California workers compensation case). In some implementations, to be able to access a case, a user must be logged in as a user that is associated with a customer account that is a valid party to a case managed by document management process 10. As will be discussed in greater detail below, case parties may be permitted to create bundles, share bundles, add and remove case records and private documents from bundles, view and download bundle documents, and view bundle tracking information.

In some implementations, a case participant may be an individual who, while not a party to a case, has been granted access to limited information related to a case by an user who is a valid case party. In some implementations and as will be discussed in greater detail below, when a case party shares a bundle with a case participant, document management process 10 may create a user account for the participant and forwards login credentials via e-mail. In some implementations, case participants may be permitted to add and remove private documents from bundles and to view and download bundle documents.

As will be discussed in greater detail below, document management process 10 may create, edit, and/or delete bundles of case records and/or private documents; allow users to add and/or remove case records and private documents to bundles; share bundles with case parties and participants; allow users to view and/or download bundle documents; automatically add case documents to bundles (i.e., live bundles); create authorized medical review bundles; create indexed bundles; create and file exhibit bundles; automatically track bundles and bundle documents; and/or highlight bundles that have recently been updated.

In some implementations, document management process 10 may create, edit, and/or delete bundles of case records and/or private documents. As discussed above, bundles may generally be collections of case-related documents created for specific purposes (e.g. highlighting case arguments, sharing documents for medical review, etc.). In some implementations, case parties may be permitted to create bundles. A case party may generally include the parties associated with a matter. For example, the case party may include the applicant, defendant, legal representatives (e.g. applicant and defense attorneys), and/or claims administrators associated with a legal matter. In some implementations, the owner (i.e. creator) of a bundle may be permitted to edit or delete the bundle.

Referring also to the example of FIG. 3 and in some implementations, a user may select a particular button of a user interface generated by document management process 10 to create a bundle (e.g., a Create Bundle button). A user may then provide a title and description for the bundle. To complete the bundle, the user may select a save button.

Referring also to the example of FIG. 4 and in some implementations, a user may select a particular bundle from a plurality of bundles displayed on a user interface generated by document management process 10 (e.g., a dropdown list of bundles). In some implementations, a user may be provided with an Edit Bundle Details icon from a toolbar. In some implementations, the Edit Bundle Details may change color and document management process 10 may generate the Edit Bundle Detail form (as shown in FIG. 4). A user may provide a title for the bundle via a title input box and a description for the bundle. After the user has completed editing, the changes may be saved by selecting a save button.

In some implementations, a user may select a particular bundle from a plurality of bundles displayed on a user interface generated by document management process 10 (e.g., a dropdown list of bundles). In some implementations, a user may be provided with a Delete Bundle icon from a toolbar. In some implementations, a user selection of the Delete Bundle icon may cause document management process 10 to render a confirmation dialog box from which a user may select an option to proceed with the deletion of the bundle or an option to cancel the deletion of the bundle.

In some implementations, document management process 10 may share one or more bundles with case parties and/or participants. For example, the creator of a bundle may be able to share the bundle with other parties to the case. In some implementations, the creator of a bundle may be able to share the bundle with non-case individuals and organizations (participants) by providing contact information. For example, when a bundle is shared with a participant who does not already exist in the system, a new user account may be created for the participant and login information may be sent to them via e-mail.

Referring also to the example of FIG. 5 and in some implementations, document management process 10 may allow a user to share bundles with other individuals and/or entities. For example, document management process 10 may allow a user to share bundles with case parties. As discussed above, a case party may generally include the applicant, defendant, legal representatives (e.g. applicant and defense attorneys), and/or claims administrators associated with a case (e.g., a California workers compensation case). In some implementations, a user may select a particular bundle from a plurality of bundles displayed on a user interface generated by document management process 10 (e.g., a dropdown list of bundles). The user may select a Share Bundle icon from a toolbar. In this example, the Share Bundle icon may change color and document management process 10 may generate a Share Bundle form. In some implementations, the user may be provided with options of case parties to share the bundle with. In the example of FIG. 5, the selection icon next to each party may be a toggle. Repeatedly clicking on the selection icon of any party may alternately change its state between selected and deselected. Selected items may be indicated by a selection icon that contains a checkmark. The bundle may only be shared with selected parties.

In some implementations, document management process 10 may allow a user to share bundles with case participants. As discussed above, a case participant may generally include an individual who, while not a party to a case, has been granted access to limited information related to a case by an user who is a valid case party. In some implementations, a user may select a particular bundle from a plurality of bundles displayed on a user interface generated by document management process 10 (e.g., a dropdown list of bundles). The user may select a Share Bundle icon from a toolbar. In this example, the Share Bundle icon may change color and document management process 10 may generate a Share Bundle form. If the participant that the user would like to share the bundle already exists in the “Share collection with selected parties/participants” area of the form, the user may continue as discussed above. If the participant that the user would like to share the bundle with does not exist in the “Share collection with selected parties/participants” area of the form, the user may add the participant to the case as follows. The user may enter the participant's First Name, Last Name, and company E-Mail address into the appropriate input boxes in the “Share with a third party participant” area of the form. The user may click on the Add to List button. If the participant does not already exist, document management process 10 may automatically generate a system user using the entered E-Mail address as the username and a random password. In this example, an e-mail may be sent to the participant with their login credentials and instructions on how to access the system. In this example, the participant may be added to the list in the “Share collection with selected parties/participants” area of the form. The user may continue as described above.

In some implementations, document management process 10 may allow users to add or remove case records and private documents to bundles. For example, all users who have access to a bundle (i.e. by virtue of ownership or sharing) may be permitted to add case records they own to a bundle. in another example, all users who have access to a bundle may be permitted to add private documents to a bundle. In some implementations, users may not be permitted to add case records that they do not own to a bundle. Users may not be permitted to add private documents that have been shared with them by others to a bundle. In some implementations, users may not be permitted to remove case records and private documents from a bundle that they have added themselves. Users may not be permitted to remove case records and private documents from a bundle that another user has added. In some implementations, when a user removes a private document from a case, the document may be automatically removed from all bundles in the case that it has been included in.

Referring also to the example of FIG. 6 and in some implementations, document management process 10 may allow a user to add documents to a bundle. For example, a user may select a particular bundle from a plurality of bundles displayed on a user interface generated by document management process 10 (e.g., a dropdown list of bundles). In some implementations, the user may select an Add Documents icon from a toolbar. In this example, the Add Documents icon may change color and document management process 10 may generate a list of case records and the user's private documents may be shown to the user. The user may select on a particular icon (e.g., in the left column) to select a document to be added to a bundle. In some implementations, the selection icon may change to a box with a checkmark in it to indicate that the document will be added to the bundle. In some implementations, the selection icon in the left column may be a toggle. Repeatedly clicking on the selection icon of any document may alternately change its state between selected and deselected. Selected items may be indicated by a selection icon that contains a checkmark. Selected items will be added to the bundle. Non-selected items may not be added to the bundle.

In some implementations, document management process 10 may allow a user to remove documents from a bundle. For example, a user may select a particular bundle from a plurality of bundles displayed on a user interface generated by document management process 10 (e.g., a dropdown list of bundles). In some implementations, the user may select a Remove File from Bundle icon from a toolbar. In this example, the Remove File from Bundle icon may change color and document management process 10 may generate confirmation box to which user may confirm approval to remove the selected file form the bundle or cancel the operation.

In some implementations, document management process 10 may allow users to view and/or download bundle documents (i.e., documents corresponding to a particular bundle). For example, all users who have access to a Bundle (i.e. by virtue of ownership or sharing) may be permitted to view the entire contents of a Bundle. In some implementations, all users who have access to a bundle may be permitted to view case records that they own. in some implementations, all users who have access to a bundle may be permitted to view all private documents that belong to the bundle. All users who have access to a bundle may be permitted to download case records that they own. In some implementations, all users who have access to a bundle may be permitted to download all private documents that belong to the bundle. In some implementations, all case parties who have access to a bundle are permitted to purchase any case records belonging to the bundle that they do not own. Once purchased, the case record may be available to view or download from the user's My Documents list and from all bundles that contain it.

Referring also to the example of FIGS. 6-7 and in some implementations, document management process 10 may allow users to view and/or download bundle documents (i.e., documents corresponding to a particular bundle). For example, a user may select a particular bundle from a plurality of bundles displayed on a user interface generated by document management process 10 (e.g., a dropdown list of bundles). In some implementations, a user may select a name of an available document to open. In some implementation, document management process 10 may generate a new tab or window of a user's browser with a document viewing application including the selected document. Similarly, document management process 10 may allow a user to download a bundle from a plurality of bundles displayed on the user interface generated by document management process 10. A user may select a Download icon associated with a particular document to download the document.

In some implementations, document management process 10 may automatically add case documents to bundles (i.e., live bundles). For example, the creator of a bundle may designate that new case records that are obtained by another application or service (e.g., Med-Legal) on behalf of a case party to be automatically added (e.g., via document management process 10) to the bundle's list of documents. In some implementations, the creator of a Bundle may configure the bundle to include only new records that match a pre-selected set of criteria (e.g. new Medical Records only). When a new case record is obtained by another application or service (e.g., Med-Legal) that matches the live bundle criteria, document management process 10 may automatically add the document to all the My Documents list of all case parties and to all live bundles' lists. In some implementations, all users who have access to a Bundle may be able to see the automatically added case record in the bundle's documents list. In some implementations, only users who have purchased a copy of the record displayed in the live bundle from another application or service (e.g., Med-Legal) may be granted the ability to view or to download the record. In some implementations, case parties who have access to a live bundle may be permitted to purchase any automatically added case record. For example and once purchased, the case record may be available to view or download from the user's My Documents list and from all bundles that contain it.

In some implementations, document management process 10 may interact with a bundle update service (e.g., the Med-Legal Bundle Update Service). For example, the bundle update service (e.g., the Med-Legal Bundle Update Service) may be an electronic agent that identifies case records that ensures that live bundles include all of the records ordered for a case that the application (e.g., Med-Legal) has access to. For example, on a daily basis, the bundle update service may add every new record that has been delivered in the previous day to every live bundle that the record can be included in based upon case and bundle new record criteria.

Referring also to the example of FIG. 8 and in some implementations, document management process 10 may automatically add case documents to bundles (i.e., live bundles). For example, a user may select a Create Bundle button. The user may then enter a title and description. A user may then be provided with an indicator to reflect that case records should be automatically added by document management process 10 to the bundle. Upon saving the bundle, document management process 10 may automatically add particular case documents to bundles.

In some implementations, document management process 10 may create authorized medical review bundles. For example and via document management process 10, users may create authorized medical review bundles. An authorized medical review bundle may have additional properties that make them suitable for use in Independent Medical Review (IMR). In some implementations, a user who creates an authorized medical review bundles may be required to select an Independent Medical Review provider associated with the bundle. In some implementations, an authorized medical review bundle may enforce a workflow where case parties approve the contents of the bundle, the bundle is forwarded to the Independent Medical Review provider, and the results of the Independent Medical Review are uploaded to the bundle.

Referring also to the example of FIG. 9 and in some implementations, document management process 10 may create authorized medical review bundles. As discussed above, an authorized medical review bundle may have additional properties that make them suitable for use in Independent Medical Review (IMR). In one example, a user may select a Create Bundle button. The user may then enter a title and description for the bundle. In some implementations, the user may select an authorized medical review from the bundle type dropdown list control. In some implementations, a user may select their desired Qualified Medical Examiner (QME) from the authorized medical reviewers dropdown list control generated by document management process 10. The user may select the Add Documents icon from a toolbar. In some implementations, the Add Documents icon may change color and a list of case records and the user's private documents may be shown. The user may select a selection icon (e.g., in the left column) to select a document to be added to the bundle. The selection icon may change to a box with a checkmark in it to indicate that the document will be added to the bundle. The selection icon (e.g., in the left column) may be a toggle. Repeatedly clicking on the selection icon of any document may alternately change its state between selected and deselected. Selected items may be indicated by a selection icon that contains a checkmark. Selected items may be added to the bundle while non-selected items may not be added to the Bundle. In some implementations, the changes to the bundle may be saved.

In some implementations and continuing with the above example, the user may select on the Approve Documents button in the toolbar to notify all parties to the case that the documents are available for review. Once the bundle has been approved for medical review, the bundle may be locked from further changes by the parties. In some implementations and depending upon the Medical Review Service selected, document management process 10 may automatically export the Medical Review bundle's documents into the Medical Review Service's system for review. Some services may require the Medical Reviewer to log in to document management process 10 to download the bundle's documents into their system. In some implementations and depending upon the Medical Review Service selected, document management process 10 may automatically import the Medical Review Services Report(s) into the medical review bundle documents listing when they have been completed. Some services will require the Medical Reviewer to log in to document management process 10 to upload the reports to the Medical Review Bundle. In some implementations, document management process 10 may add the Medical Reviewer's reports to medical review bundle. Case parties may be permitted to review the Medical Reviewer's report(s) directly from the medical review bundle interface.

In some implementations, document management process 10 may create indexed bundles. For example and via document management process 10, users may create indexed bundles. In some implementations, an indexed bundle may include an ordered list of active annotations to the bundles' documents. Each active annotation may be composed of some annotation text and a link to a specific location in one of the bundles' documents. In some implementations, the owner of an indexed bundle may add, edit, remove and re-order active annotations within the bundle. All users who have access to an indexed bundle may be able to view the list of annotations associated with the bundle. In some implementations, the list of annotations may contain each item's annotation text, the document and page referenced, and an affordance to the associated page. For example, each active annotation affordance may be styled differently based upon whether a user has access to the referenced bundle's document or not. In some implementations, all users who have access to an indexed bundle may be able to activate the active annotation's links for bundle documents that they have access to. For example, when an active annotation's affordance is selected, the associated bundle document may be opened in the document viewer and the viewer is advanced to the appropriate location within the document. In some implementations, when a user who has access to an indexed bundle attempts to active an active annotation's affordance for a bundle document that they do not have access to, the user may be provided with a mechanism to purchase access to the associated document. Once purchased, the user may have access to the document in their My Documents list and in every bundle that contains the document.

Referring also to the example of FIG. 10 and in some implementations, document management process 10 may create indexed bundles. As discussed above, an indexed bundle may include an ordered list of active annotations to the bundles' documents. In some implementations, a user may select a Create Bundle button. The user may then enter a title and description for the bundle. In some implementations, the user may select an indexed bundle from the bundle type dropdown list control. In some implementations, the user may select an Add Documents icon from a toolbar. In this example, the Add Documents icon may change color and document management process 10 may generate a list of case records and the user's private documents may be shown to the user. The user may select on a particular icon (e.g., in the left column) to select a document to be added to a bundle. The user may select on a particular icon (e.g., in the left column) to select a document to be added to a bundle. In some implementations, the selection icon may change to a box with a checkmark in it to indicate that the document will be added to the bundle. In some implementations, the selection icon in the left column may be a toggle. Repeatedly clicking on the selection icon of any document may alternately change its state between selected and deselected. Selected items may be indicated by a selection icon that contains a checkmark. Selected items will be added to the bundle. Non-selected items may not be added to the bundle.

In some implementations, document management process 10 may allow a user to add comments to an indexed bundle. For example, document management process 10 may allow a user to select an indexed bundle from the bundle dropdown list. The user may select a View Index icon from the toolbar. In some implementations, the View Index icon may change colors and a list of index comments may be shown. The user may then select an Add Index Comment control to add a new index comment. In some implementations, document management process 10 may display an Add Index Comment form. The user may enter the index comment in an Index Comment Edit control. The user may select the bundle document associated with the index comment from the bundle document's dropdown list control. In some implementations, document management process 10 may add the page number from the bundle document associated with the index comment in a page number edit control. The changes may be saved and the list of index comments may be updated to reflect the user's changes.

In some implementations, document management process 10 may allow a user to view documents associated with index bundle comments. For example, document management process 10 may allow a user to select an indexed bundle from the bundle dropdown list. The user may select a View Index icon from the toolbar. In some implementations, the View Index icon may change colors and a list of index comments may be shown. In some implementations, the user may select a View Document Location icon next to the desired Index Comment. Document management process 10 may add a new tab to the user's browser with a document viewer containing the selected document. If the index comment has a page number associated with it, the document viewer may navigate to the appropriate page.

In some implementations, document management process 10 may allow a user to remove comments from an indexed bundle. For example, a user may select an indexed bundle from the bundle dropdown list. In some implementations, the user may select a View Index icon from the toolbar. In some implementations, the View Index icon may change color and a list of index comments may be shown. In some implementations, the user may select a view document location icon next to the desired index comment. Accordingly, the index comment may be removed from the list of index comments.

In some implementations, document management process 10 may allow a user to reorder indexed bundle comments. For example, a user may select an indexed bundle from the bundle dropdown list. In some implementations, the user may select a View Index icon from the toolbar. In some implementations, the View Index icon may change color and a list of index comments may be shown. The user may then select the index comment's handle and may drag it to the desired position in the list.

In some implementations, document management process 10 may create and file exhibit bundles. For example and via document management process 10, users may create exhibit bundles. An exhibit bundle may generally enforce a workflow that permits the user to file the documents contained in the bundle as an exhibit to a case.

Referring also to the examples of FIGS. 11-12 and in some implementations, document management process 10 may allow a user to file an exhibit bundle. For example, a user may select a My Filings view from a case views dropdown list. The user may select a Create New Filing control. In some implementations, the user may select File Exhibit from the filing types popup list control. In some implementations, document management process 10 may display the File Exhibit form. The user may enter a title and a description for the bundle. In some implementations, the user may select the Add Document to Exhibit icon from a toolbar. In some implementations, document management process 10 may display an Add Document form. The user may select a Document Type from a Document Type dropdown list control and the user may select a Document Title from a Document Title dropdown list control. In some implementations, the user may select the Document from the case documents dropdown list control. The user may optionally change the date in the Document Date control. The user may select the Add Document button to add the document to the Exhibit or may cancel the operation. Document management process 10 may return the user to the File Exhibit form. In some implementations, the user may continue to Add Documents as discussed above until they are complete. Following the user selection, the user may select the File Exhibit button to file the exhibit. Document management process 10 may generate the necessary document to file the exhibit and add it to a list of case documents. In some implementations, document management process 10 may file the exhibit electronically and may notify the other case parties.

In some implementations, document management process 10 may interact with a filing engine or service (e.g., the Med-Legal Filing Engine). For example, the filing engine (e.g., the Med-Legal Filing Engine) may be an electronic agent that performs a number of operations necessary to file cases with a particular entity (e.g., the California Division of Worker's Compensation Electronic Adjudication Management System (EAMS)). In some implementations, document management process 10 may interact with the filing engine to file exhibits for exhibit bundles.

In some implementations, document management process 10 may automatically track bundles and bundle documents. For example, document management process 10 may records information for the following events: bundle creation (i.e., user, date and time); bundle deletion (i.e., user, date and time); bundle share added (i.e., user, date and time, share party/participant); bundle share removed (i.e., user, date and time, share party/participant); document added to bundle (either manually or automatically) (i.e., user, date and time, document); document removed from bundle (i.e., user, date and time, document); bundle opened (i.e., user, date and time; bundle document viewed (i.e., user, date and time, document); and bundle document downloaded (i.e., user, date and time, document). While examples have been provided of the types of events and type of information that may be recorded for the various events, it will be appreciated that any type of event and any information about that event may be recorded and tracked by document management process 10. In some implementations, all case parties who have access to a bundle may view the bundle's tracking information.

In some implementations, document management process 10 may highlight bundles that have been recently used. For example, document management process 10 may provide a dashboard display to a user with a list of the e.g., 5 cases that have bundles that the user has access to that have been recently shared or updated. In some implementations, when the user selects the list's title, document management process 10 may navigate to the cases listing with the “Cases With Recently Shared Bundles (Last 90 Days)” filter selected. In some implementations, when the user selects an item from the list, document management process 10 may navigate to the case details page for the case containing the bundle. In some implementations, when the user selects the “Cases With Recently Shared Bundles (Last 90 Days)” filter from the cases listing, document management process 10 may display a paginated list of all cases that the user has access to that have been recently shared or updated. When the user selects the case number for a case in the listing, document management process 10 may navigate to the case details page for the case.

### Active Case Parties

In some implementations, document management process 10 may provide a current, immediate, and verified view to active and authorized case parties for an adjudicated case (e.g., within the California Workers' Compensation System (CWCS)). Identifying, knowing, and maintaining such case parties may be an absolute requirement to completely and properly process discovery and related information/requests (e.g., within the CWCS). Embodiments of the present disclosure may allow case parties to be created, identified, and maintained using a proprietary methodology that (best) matches (and refines) input/user specified names to system-authorized naming conventions by processing the input/provided name on a fuzzy-logic basis against an independently generated list of possibilities based on entity reference number (ERN) conventions within a predefined directory (e.g., the CWCS Electronic Adjudication Management System (EAMS)), ultimately best-matching such conventions to the most likely intended case party name, and (in turn) connecting the match thereafter to only a verified Uniformed Assigned Name (UAN) as is required for a case party.

In some implementations, the case party information may be constantly reviewed and updated, based on any/all party input/interaction—providing the most up to date view of such case party information. Additionally, the fuzzy-logic connection and related processing to entity reference number (ERN) names (and numbers) that ultimately best-matches to the likely case party, may provide a level of accuracy unavailable (except by manual search and revision) in any other system. Finally, by connecting the most-likely name (from ERN processing) to the required/absolute name (from UAN processing) assures the accuracy/integrity of the case party information to the very precise requirement of various systems (e.g., the CWCS).

In some implementations, various relevant matters may be managed by various electronic systems. For example, California workers compensation cases may be managed in the Electronic Adjudication Management System (EAMS), a computer-based case management system. In some implementations, EAMS may maintain an electronic record of the workers compensation cases that have been filed. As workers compensation cases work their way through the legal process, EAMS may be updated to reflect the cases current state. EAMS may maintain a list of legal representatives (e.g. applicant and defense attorneys), claims administrators, and lien claimant accounts that are associated with the cases in EAMS. These entities may be identified by both a numerical EAMS Reference Number (ERN) and a descriptive Uniformed Assigned Name (UAN). However, the parties associated with a case in EAMS may frequently change over the life of the case.

In some implementations, various applications or services may maintain case information separately from other electronic systems (e.g., EAMS). For example, Med-Legal maintains its own local electronic data store of case information. This case information included the ERN and UAN of authorized case parties as reported by EAMS. In some implementations, a synchronization engine (e.g., the Med-Legal EAMS Synchronization Engine) may automatically synchronize the separate case parties (e.g., Med-Legal Authorized Case Parties Store with EAMS) on a regular basis.

In some implementations, various applications may employ customer relationship management systems. For example, Med-Legal maintains a Customer Relationship Management (CRM) system containing information regarding its customers and the systems that they are authorized to access. Med-Legal's CRM may distinguish between users and the companies that they work for. Users represent people who are employed on behalf of a company to perform work on its behalf. For example and for every customer, Med-Legal CRM may maintain the company's EAMS Reference Number (ERN), Uniform Assigned Name (UAN), and a list of the users that are authorized to access Med-Legal's systems on their behalf. In certain circumstances, a user may be authorized to perform work for multiple entities, each having a separate ERN and UAN.

In some implementations, document management process 10 may interact with various synchronization engines. For example, a synchronization engine (e.g., the Med-Legal EAMS Synchronization Engine) may be an electronic agent that downloads the current set of data for a case from EAMS, performs a series of calculations and transformations on the data, and imports the data into a particular data store (e.g., the Med-Legal Authorized Case Parties Data Store). In some implementations, the Med-Legal EAMS Synchronization Engine may compare the current state of the case information in EAMS with the Med-Legal Authorized Parties Data Store and records additional information to identify when a party is added or removed from a case. In order to improve system and network efficiency, the Synchronization Engine may maintain a queue of cases which will be synchronized with EAMS. The ability to specify the criteria with which cases are added to the queue may be dynamic. For example, current criteria may include 1) a customer request for a new record which does not have case information, 2) the number of days since the case was last synchronized with EAMS, and an internal request for a case update. Additional criteria may generally include 1) a customer request to be added to a case, 2) the creation of an internal work order for work to be performed on behalf of a case, and 3) the generation of an invoice related to a case.

Referring also to the example of FIG. 13 and in some implementations, document management process 10 may synchronize case parties across various data stores. For example, a EAMS Case Party Synchronization service may run as a headless application that is launched according to a predetermined schedule. The service may log in to the EAMS portal. The service may read the next case from the case synchronization queue. In some implementations, the next case may be determined by case request urgency followed by the time that the request was made on a first in/first out (FIFO) basis. If a case was not retrieved, the service waits a pre-determined amount of time then proceeds with the next case from the synchronization queue. If a case was retrieved, the service may read the current list of authorized case parties from the Med-Legal authorized case parties data store. If there is no record of the case in the Med-Legal authorized case parties data store, an empty list is created. The service may read the current list of case parties from EAMS. The service may compare the authorized case parties list with the EAMS Case Parties list and: 1) add all case parties that exist in EAMS that are not in the authorized case parties list. For each case party, set the ADDED ON field to the current date; 2) for all case parties that exist in the authorized case parties list that do not exist in EAMS, set the DELETED_ON field to the current date; and 3) for all case parties that exist in both EAMS and the authorized case parties lists but where the case party information has changed, set the MODIFIED_DATE to the current date.

In some implementations, document management process 10 may set the authorized case parties list LAST_UPDATED field to the current date. The updated case parties list may be saved to the Authorized Case Parties Data Store, over-writing the previous state of the list. Document management process 10 may compare the number of times that case party information has been accessed from EAMS during the current day with the pre-determined limit of the number of time case party information should be accessed from EAMS. If the number of times that case party information has been accessed from EAMS is less than or equal to the number of times case party information should be accessed from EAMS, the service may read the next case from the synchronization queue. If the number of times that case party information has been accessed from EAMS is greater than the number of times case party information should be accessed from EAMS, document management process 10 may terminate the EAMS Case Party Synchronization service.

In some implementations, document management process 10 may allow customer on-boarding. For example, a customer may complete on-boarding form which includes the company's EAMS Reference Number (ERN), Uniform Assigned Name (UAN), as well as a list of the company's employees that are permitted access to document management process 10 (e.g. users). A Med-Legal Customer Service representative may validate the company's EAMS Reference Number and Uniform Assigned Name against EAMS. The Med-Legal Customer Service representative may enter the company and employee information into the Med-Legal Customer Relationship Management (CRM) system. The Med-Legal Customer Relationship Management system may send an e-mail to each of the customer account's users containing their system credentials.

In some implementations, document management process 10 may validate customer case authorization. For example and via document management process 10, a user may navigates to the portal login page associated with document management process 10 using an Internet browser. The user may provide their system credentials in the form of a username and password to the login page. The login page may call the Med-Legal Application Programming Interface (API) Gateway's login service passing the user's system credentials. The login service may authenticate the credentials against the Med-Legal Customer Relationship Management system. If the login service is not able to authenticate the user, the login service may respond to the login request with an HTTP 401 (Unauthorized) return code and the login page reports the error to the user. If the login service is able to authenticate the user, the login service may generate a JSON Web Token (JWT) containing the user's Session information, including the Account ID of the company that they are employed by. The JWT may be signed with a Med-Legal private key to prevent tampering. The portal associated with document management process 10 may store the user's Session JWT in private memory. As part of the user's interaction with the portal, the portal may call services on the Med-Legal System API Gateway using HTTP requests. When calling a service, the portal may retrieve the user's Session JWT from private memory and add it to the HTTP headers accompanying the request. The System API Gateway associated with document management process 10 may validate the request's Session JWT. If the Session JWT is invalid or expired, The System API Gateway associated with document management process 10 may return an HTTP 401 (Unauthorized) return code and the portal may remove the user's Session JWT from private memory. The portal may navigate the user to the portal login page. If the Session JWT is valid, the System API Gateway service computes a response to the request is in accord with the requesting user's authorizations by comparing the EAMS Reference Number associated with the operation with the EAMS Reference Number associated with the account ID contained in the Session JWT. The System API Gateway associated with document management process 10 returns an HTTP 200 (Success) return code. The portal continues with the user's requested operation.

### Active Contenting

In some embodiments, document management process 10 may provide a unique capability to work ‘within’ the discovery/evidence to make it more usable, visible, and relevant. In particular, document management process 10 may process the evidentiary content available following a proprietary outline/format that specifically categorizes particular evidentiary elements that apply in relevance to the case being adjudicated; including excerpting (and onward indexing) of key (identified) evidentiary elements within the evidence. Further, the processing can be either static (standard format/input/output) or dynamic (unique input elements, characteristics, or combined in chronology) to provide a comprehensive and/or combined view of the history, progression, and status of the medical condition (represented within the evidence). In turn, this information is easily extracted into required medical reports and/or analysis that is required as part of most cases (e.g., any California Workers' Compensation System (CWCS) medical-legal evaluation). Finally, specialized paths/formats are available to best-content the results for (various, as listed) medical specialties often active in such evaluations.

With document management process 10, the discovered information (evidence) becomes more visible, useful, and relevant—ultimately speeding and facilitating the understanding of complex medical conditions, in turn improving the accuracy of medical-legal evaluations required to proceed with treatment—or to onward confirm or deny such treatment in/for subsequent medical-legal evaluations. Additionally, that same enhanced content can be used to highlight important medical/conditional distinctions—providing a more accurate listing of key conceptual constructs in the case, especially regarding causation, ultimately leading to more timely and right valued settlements on the case. Finally, with medical information so tightly organized and excerpted (directly within the evidence)—reports thereon can be written quicker, easier, and more accurately (with clear reference to the underlying medical record).

In some implementations, case records are often long collections of partially related documents that are littered with technical terminology which make it difficult for the uninitiated to follow. Generally, case records are reviewed manually following a proprietary checklist of review topics to ensure completeness and consistency. Review results are encoded electronically in a database format suitable for electronic analysis, ad-hoc inquiry, and the generation of graphical representations of the data. Standard Review of case records includes the creation of a standard table of contents for the case record. The table of contents may expose the underlying structure of the case record which facilitates efficient document navigation and overall comprehension.

In some implementations, document management process 10 may generate one or more case record excerpts. A case record excerpt may be a listing of the essential facts contained in the document including dates of service, service provider, a summary of the pertinent fact, and a list of page numbers. In some implementations, document management process 10 may present case record excerpts in a tabular format.

In some implementations, each excerpt summary may include a list of the page numbers which refer to the page in the case record which supports the summary statement. Page numbers may be presented in the form of dynamic links which, when activated, navigate the user to the indicated page in the case record.

In some implementations, document management process 10 may generate one or more enhanced case records. An enhanced case record encodes a case record's table of contents, excerpt, and record scans into a self-contained Portable Document Format (PDF) file. The table of contents in the enhanced case record may be presented in a hyperlinked format where selecting an entry navigates the user to the specified section of the document. The excerpt in the enhanced case record may be presented in a tabular format listing the date of service, service provider, summary of pertinent fact, and a list of the page numbers associated with summary as described above. The page numbers are represented in a hyperlinked format where selecting an entry navigates the user to the specified page of the document.

In some implementations, document management process 10 may provide a chronological view of the case, a tabular representation of facts of the case in a tabular format which includes the date, item description, case record title, and case record type. The case record titles may be presented in a hyperlinked format which, when selected, navigate the user to the first page of the enhanced case record. The chronological view may be a comprehensive listing of the dates reported for the case (e.g., in the California Division of Workers Compensation Electronic Adjudication Management Systems (EAMS)), the fact summaries from all of the excerpts compiled for the records in the case, and the service dates for the records in the case that do not have excerpts. The item description for excerpts may include the detail summary, service provider, and the pages in the case record supporting the item. The pages are presented in a hyperlinked format which, when selected, navigate the user to the appropriate page of the case record. The item description for non-experted records contains the service provider's name.

In some implementations, case parties and case participants may be permitted to view enhanced case records. In some implementations, a record reviewer is a service provider who reviews case records, builds a table of contents for the document based upon its underlying structure, extracts relevant fact details from the document's content, identifies additional records that may be relevant to the case, and records the information in appropriate Med-Legal systems.

In some implementations, document management process 10 may interact with a case record processing service (e.g., the Med-Legal Case Record Post Processing Service). For example, a case record processing service (e.g., the Med-Legal Case Record Post Processing Service) may be an electronic agent that performs a series of operations on records following the Record Review process. The case record processing service may generates an enhanced case record which encodes a case record's table of contents, excerpt, and record scans into a self-contained Portable Document Format (PDF) file. The Med-Legal Case Record Post Processing Service, for example, may perform computation based on excerpts contained in the case RAML file and may update the Med-Legal Case Analytics database with the relevant information.

In some implementations, document management process 10 may provide a case record for record review. For example, document management process 10 may deliver a new case record to a record reviewer. As discussed above, the Record Reviewer may generate the table of contents for the case records and encodes the information in a Record Annotation Markup Language (RAML) file. The record reviewer may review the case record for relevant facts based upon the record review checklist. The Facts may be added to the RAML file for the record. When additional records that are potentially relevant to the case are identified during record review, the information may be added to the RAML file for the record and Med-Legal's Complete Medical History Administration System.

Referring also to the examples of FIGS. 14-15 and in some implementations and as discussed above, document management process 10 may generate one or more enhanced case records. For example, document management process 10 may interact with a case record post processing service. The case record post processing service may retrieve the next record to process from the enhanced case record generation queue based upon e.g., a first in first out (FIFO) methodology. The case record post processing service may create a new Portable Document Format (PDF) file for the Enhanced Case Record. The case record post processing service may open the Record Annotation Markup Language (RAML) file for the record. In some implementations, the case record post processing service may retrieve the table of contents from the RAML file for the Record and generates a table of contents. The case record post processing service may retrieve the list of excerpt facts from the RAML file for the record and generates the excerpt table. The case record post processing service may append the original case record to the enhanced case record. The case record post processing service may iterate across the table of contents entries and, for each entry, converts the entry into a link to the relevant page in the document. The case record post processing service may iterate across the excerpt table. Document management process 10 may iterate across each page number listed in the pages field of the table and, for each page number, converts the page number into a link to the relevant page in the document.

In some implementations, document management process 10 may provide analytics associated with the case records. For example, document management process 10 may, via the case record post processing service, retrieve the next record to process from the enhanced case record generation queue based upon a first in first out (FIFO) methodology. The case record post processing service may open the Record Annotation Markup Language (RAML) file for the record. The case record post processing service may update the Med-Legal Enhanced Case Record Details database with the record's excerpt details to support the case's chronological view. The case record post processing service may perform computations based on excerpts contained in the case RAML file and may update the Med-Legal Case Analytics database with the relevant information.

### Active Discovery

In some implementations, document management process 10 may provide a current, immediate, and verified view to authorized discovery that is available, active, underway, or possible for/from all case parties for an adjudicated case (e.g., within the California Workers' Compensation System (CWCS)). Identifying, knowing, accessing, maintaining, and using such authorized discovery may be desirable to completely and properly administer, process, adjudicate cases within an ‘evidence-based’ system of medical care (e.g., within the CWCS). In some implementations, document management process 10 may provide a means to more deeply identify all of the required evidence (either in form of a Complete Medical History (CMH) or Rated Medical History (RMH)) by evaluating (identified/all of) the discovery already available/complete against any of; 1) an absolute list of additional discovery options identified therein (CMH), 2) a static-based ranking (heat-map) of that (same) absolute list matching to a regular, defined, continuing list of prioritization (SRMH), or 3) a dynamic-based ranking (heat-map) allowing dynamic adjustment of defined specification to improve the (relative) prioritization of available discovery within the otherwise available absolute list (DRMH).

Accordingly, the discovered information (evidence) may be constantly renewed, supplemented, and updated, based on any/all party input/interaction—providing the most up to date view of available evidence for a case. Additionally, document management process 10 may enable immediate and timely access to newly produced discovery such that additional available evidence is constantly visible and updated in the ‘availability dashboard’—providing the capability to purchase full access to such discovery in real-time. Further, with available heat-mapping capabilities driven by lists that are either absolute, static, or dynamic; the evidence available may become visible and ranked in importance to the case (or administration or adjudication parameters) thereon.

Referring also to the example of FIG. 16 and in some implementations, document management process 10 may allow case parties to review complete medial history. For example, document management process 10 may provide case parties with a listing of potential records related to a case that have not been ordered via a Complete Medical History (CMH) Portal or other medical history source. The Complete Medical History Portal may generally include a collection of user interfaces that allow a user to search and order records that have not yet been acquired. Case parties may order records recommended by the Complete Medical History Portal using a streamlined ordering process. Case parties may ignore records recommended by the Complete Medical History Portal. Ignored records may be removed from the Complete Medical History Portal. Case parties may restore ignored records recommended by the Complete Medical History Portal. Restored records may be visible in the Complete Medical History Portal. In some implementations, a Rated Medical History (RMH) Portal may be a collection of user interfaces that allow a user to evaluate the evidentiary usefulness of the records recommended by Complete Medical History. The RMH Portal may allow a user to filter, search, and order records that have not yet been acquired.

In some implementations, document management process 10 may provide two mechanisms to assist case parties in evaluating the evidentiary usefulness of the records recommended by Complete Medical History: static rated medical history and dynamic rated medical history. For static rated medical history, each record recommended by the Complete Medical History Portal is compared against a static list of factors during standard processing and a numerical value is calculated for each factor representing how valuable the record is with respect to that factor. Common static factors may include timeliness, record types, significant keywords, etc. For dynamic rated medical history, each record recommended by the Complete Medical History Portal may be compared against a dynamic list of factors that the user provides at run time. A numerical value may be calculated immediately for each user-entered factor representing how valuable the record is with respect to that factor. Document management process 10 may provide dynamic factors for location and keywords. In some implementations, rated medical history may be presented via a heat map of existing records in tabular form to provide the user with a visual indicator of the potential evidentiary value of the record to the case. Each cell in the resulting matrix of records and factors may include the calculated numerical value of the factor for the record and is assigned a color based upon the size of that value. Case parties may order records in the static rated medical history view by selecting an “order” button for the record.

In some implementations, case parties may be permitted to order records recommended by Complete Medical History. As discussed above, a record reviewer may be a service provider who reviews case records and identifies additional records that may be relevant to the case but have not been ordered. The record reviewer may enter the information in appropriate Med-Legal systems.

In some implementations, document management process 10 may interact or interface with a medical history data store (e.g., the Complete Medical History (CMH) data store). For example, the Complete Medical History (CMH) data store may be a repository of information related to complete medical history and rated medical history. The CMH data store may maintain information for every additional record that a record reviewer has identified that may be relevant to the case including the type of record, the date of service for the record, and the details of record's provider. The Med-Legal Complete Medical History Services may receive information entered by the record reviewers and perform the necessary calculations to ensure that the medical history data store (e.g., the Complete Medical History data store) has the most recent information and is complete.

In some implementations, document management process 10 may interact with one or more medical history services (e.g., the Med-Legal Complete Medical History Service). For example, a medical history service (e.g., the Med-Legal Complete Medical History Service) may be an electronic agent that imports the current set of Complete Medical History data for a record from the Record Review, performs a series of calculations and transformation on the data, and imports the data into the medical history data store (e.g., the Med-Legal Complete Medical History Data Store). In some implementations, the one or more medical history services (e.g., the Med-Legal Complete Medical History Service) may be responsible for calculating numerical values for each static rated medical history factor every record that it processes and ensuring that it is recorded in the Complete Medical History data store.

Referring also to the example of FIG. 16 and in some implementations, document management process 10 may search for complete medical history records. For example, a user may navigate to a Complete Medical History Portal. The user may select a search input control. The user may enter a case number, control number, applicant, defendant, facility name, address, etc. The user may select the Search button control. The Complete Medical History Portal may filter the list based upon the Search string entered.

In some implementations, document management process 10 may allow a user to order complete medical history records. For example, the user may navigate to the Complete Medical History Portal. The user may locate the record that they would like to order. The user may select the Add to Cart button control next to the record that they would like to order. The user may repeat this process for every record that they would like to order. The user may navigate to the Shopping Cart to complete the order.

In some implementations, document management process 10 may hide records from the complete medical history. For example, a user may navigate to the Complete Medical History Portal. The user may locate the record that they would like to hide. The user may select the hide icon control next to the record that they would like to order. The Complete Medical History Portal may remove the indicated record from the Complete Medical History default view.

In some implementations, document management process 10 may restore records to the complete medical history. For example, a user may navigate to the Complete Medical History Portal. The user may select the Hidden Records Filter from the Complete Medical History list of filters. The user may locate the record that they would like to restore. The user may select the Restore icon control next to the record that they would like to order. The Complete Medical History Portal removes the indicated record from the Complete Medical History Hidden Records view. The record may now be visible in the Complete Medical History default view.

Referring also to the example of FIG. 17 and in some implementations, document management process 10 may interact with one or more medical history services (e.g., Med-Legal Complete Medical History Service). For example, the medical history service (e.g., Med-Legal Complete Medical History Service) may run as a headless application that is launched according to a predetermined schedule. Document management process 10 may determine if a CMH Review data file is contained in the record review FTP Portal. If there are no CMH Review data files in the Record Review FTP Portal, document management process 10 may terminate the Med-Legal Complete Medical History Service. If there is at least one CMH Review data file in the Record Review FTP Portal, the service may retrieve the oldest CMH Review data file contained in the Record Review FTP portal. The service may read the first record in the CMH Review data file and may calculate the values for each static rated medical history factor for the record. The service may write the information for the record to the Complete Medical History Data Store. The service may determine whether the record is the last record in the CMH Review data file. If the record is the last record in the CMH Review data file, the service may close the CMH Review Data File; delete the CMH Review Data File from the Record Review FTP Portal; and determine if another CMH Review data file is contained in the record review FTP Portal. If the record is not the last record in the CMH Review data file, the service may read the next record in the CMH Review data file and calculate the values for each static rated medical history factor for the record. The service may then write the information for the record to the Complete Medical History Data Store.

In some implementations, document management process 10 may search for one or more rated medical history records. For example, a user may navigate to the Rated Medical History Portal. The user may select the Search input control and enter a case number, control number, applicant, defendant, facility name, or address to search. The Rated Medical History Portal may filter the list based upon the search string entered by the user.

Referring also to the example of FIG. 18 and in some implementations, document management process 10 may add dynamic factors to rate medical history. For example, the user may navigate to the Rated Medical History Portal and the user may select the Add Factor button control. The user may select the factor type from the factor type list control and may enter the factor details in the factor details edit control. The user may save the factor or close the Add Factor dialog without saving the dynamic factor. Document management process 10 may create a column for the factor in the Rated Medical History Portal and may display a progress control. Document management process 10 may calculate the entered factor for each visible record in the Rated Medical History Portal and updates the appropriate cell in the factor's column. Document management process 10 may remove the progress control and may start a background thread to calculate the entered factor for the remaining records in the user's Rated Medical History Portal. The dynamic factors may be removed when the user closes the Rated Medical History Portal.

In some implementations, document management process 10 may filter rated medical history records by factor. For example, a user may navigate to the Rated Medical History Portal. A user may select a Filter Records button control. A filter list control may appear under the factor title for every RMH Factor in the RMH Portal. The user may choose the filter value for the RMH Factor that they would like to filter the RMH Records by: displaying all records (low, medium, high); displaying only records that have a medium priority or higher; and/or displaying only records that have a high priority or higher. The Rated Medical History Portal may filter the list based upon the filters entered.

In some implementations, document management process 10 may allow a user to order rated medical history records. For example, a user may navigate to the Rated Medical History Portal and may locate the record that they would like to order. The user may select an Add to Cart button control next to the record that they would like to order. The user may repeat for every record that they would like to order. The user may navigate to a shopping cart to complete the order.

### Active Evidence

In some implementations, document management process 10 may provide a current and verified capability to track, view, personally utilize, notate, add private documentation, share, as defined, and report on each and every evidentiary discovery element that is active and in use for a case by a case party (or authorized participant) for an adjudicated case (e.g., within the California Workers' Compensation System (CWCS)). In particular, adjudicating such a case, often requires sending/sharing the evidentiary record with other case parties (or authorized participant) and closely tracking the sharing and use of same; including knowing when it was sent, to whom, for what purpose—and thereafter understanding the onward use of what was sent/shared including when it was received, first opened, last opened, and tracking various status, counts, and uses thereon; all that are unique as specified to standards and regulatory and statutory requirements (e.g., regulatory and statutory requirements within the CWCS).

In some implementations, the discovered information (evidence) may be tracked and verified at every step of use, specifically to statue and regulatory requirements thereon (e.g., within the California Workers' Compensation System (CWCS))—providing easy access to required information for billing, reporting, and tracking purposes, as required in regulation and statute. Additionally, that same visibility to location, status, and use—provides a means to more proactively manage the progress of case forward to completion. Finally, with the available notations, call outs, footnotes, and highlights directly within the evidence itself, whether private or shared, the evidence becomes specifically visible to points of reference, dialogue, or conclusion—providing for a quicker, more specific drive to conclusion.

In some implementations, users may be permitted to upload private documents to their My Documents collection in a case portal associated with document management process 10. All users may be permitted to add their own private document to bundles that they have created or that have been shared with them. Users may not be permitted to add private documents that have been shared with them by others to a bundle. Users may be permitted to remove private documents from a bundle that they have added themselves. Users may not be permitted to remove private documents from a bundle that another user has added. When a user removes a private document from a case, the document may be automatically removed from all bundles in the case that it has been included in.

In some implementations, all users may be permitted to view all of the documents that they have access to in their My Documents collection including case records that they own and private documents. Users may not be permitted to view case records that they do not own. All users who have access to a bundle may be permitted to view all case records that they own that belong to the bundle. All users who have access to a bundle may be permitted to view all private documents that belong to the bundle. Users may not be permitted to view case records that belong to a bundle that they do not own.

In some implementations, all users may be permitted to download all of the documents that they have access to in their My Documents collection including case records that they own and private documents. Users may not be permitted to download case records that they do not own. All users who have access to a bundle may be permitted to download all case records that they own that belong to the bundle. All users who have access to a bundle may be permitted to download all private documents that belong to the bundle. Users may not be permitted to download case records that belong to a bundle that they do not own.

In some implementations, users may have the ability to add highlights to case records and private documents. Highlights may be visible within a Document Viewer. Users may have the ability to remove highlights that they have added. Each user may be assigned a unique highlight color by the system. When a user hovers over a highlight in the Document Viewer, a tooltip may appear identifying the user who added the highlight. Users may have the ability to hide highlights within the Document Viewer.

In some implementations, users may have the ability to add notations to case records and private documents. Notations may be visible within the Document Viewer. The Document Viewer can be configured to display notations in either call out, side note, or footnote form. Notations may include the name of the user who entered the notation. Users may have the ability to remove notations that they have added. Users may have the ability to hide notations within the Document Viewer.

In some implementations, document management process 10 may log whenever a bundle is created; whenever a case party or participant is added or removed from a bundle; whenever a case record or private document is added or removed from a bundle; whenever a bundle is opened by a user; whenever a case record is purchased, viewed, or downloaded; and/or whenever a private document is uploaded, viewed, downloaded, or removed from a case. Case parties may be permitted to view bundle tracking logs in the case portal. Case parties may be permitted to view case record and private document tracking logs in the case portal.

Referring also to the example of FIG. 19 and in some implementations, document management process 10 may allow a user to upload private documents. For example, a user may open the appropriate case in the portal. The user may select the My Documents case view and may select the Upload Document icon in the My Documents menu bar. The user may select the Select Files button and selects the files to upload and/or may drag the files to upload to the upload area under the My Documents menu bar. The user may select the Upload button to upload the files to My Document or may select the Clear button to remove the files from the upload area. Uploaded files may appear in My Documents with a document type of private documents.

Referring also to the example of FIG. 20 and in some implementations, document management process 10 may allow a user to share case records and private documents with other case parties and participants.

In some implementations, document management process 10 may allow a user to remove private documents. For example, the user may open the appropriate case in the portal associated with document management process 10. The user may select the My Documents case view. The user may select the Trashcan icon next to the private document to be removed. The user may select the Delete button to remove the file from the case or may cancel the operation. The Private Document may be removed from the My Documents case view and all bundles that it is included in.

In some implementations, document management process 10 may allow a user to view case records and/or private documents. For example, a user may open the appropriate case in the portal associated with document management process 10. The user may select the appropriate case view. The user may select the name of an available document to open it in a separate window for viewing.

In some implementations, document management process 10 may allow a user to download case records and/or private documents. For example, the user may open the appropriate case in the portal associated with document management process 10. The user may select the appropriate case view. The user may select the Download icon of an available document to download it to their computing device.

In some implementations, document management process 10 may allow a user to highlight case records and/or private documents. For example, the user may open the appropriate case in the portal associated with document management process 10. The user may select the appropriate case view. The user may select the name of an available document to open it in a separate window for viewing. The user may select the text that they would like to highlight. The user may select the Add Highlight command icon from the Document Viewer menu bar.

In some implementations, document management process 10 may allow a user to annotate case records and/or private documents. For example, the user may open the appropriate case in the portal associated with document management process 10 and may select the appropriate case view. The user may select the name of an available document to open it in a separate window for viewing. The user may select the text that they would like to annotate and may select the Add Annotation command icon from the Document Viewer menu bar. The user may enter their desired text in the annotation text control. The annotated text may be highlighted, and the annotation may be displayed according to their settings.

In some implementations, document management process 10 may allow a user to remove highlights/annotations from case records and/or private documents. For example, the user may open the appropriate case in the portal associated with document management process 10 and may select the appropriate case view. The user may select the name of an available document to open it in a separate window for viewing. The user may select the highlighted or annotated text that they would like to remove the highlight or annotation from and may select the Remove Highlight/Annotation command icon from the Document Viewer menu bar.

In some implementations, document management process 10 may allow a user to enable/disable highlights in the Document Viewer. For example, the user may open the appropriate document in the Document Viewer and the user may select the >> command icon from the Document Viewer menu bar. The user may select the Show Highlights menu item to display highlights or Hide Highlights to hide highlights. In some implementations, document management process 10 may allow a user to configure the display of annotations in a Document Viewer.

In some implementations, document management process 10 may allow a user to view bundle tracking logs. For example, the user may open the appropriate case in the portal associated with document management process 10. The user may select the appropriate bundle in the case view. The user may select the View Tracking Information in the bundle menu bar to display the bundle tracking log which may open in a separate browser tab.

Referring also to the example of FIGS. 21-24 and in some implementations, document management process 10 may allow a user to view document tracking logs. For example, the user may open the appropriate case in the portal associated with document management process 10. The user may select the appropriate case view and may select the View Tracking Information icon next to a record or private document to display its document tracking log.

### Active Settlement

In some implementations, document management process 10 may provide a consistent process, workflow, and construct to proactively settle active cases (e.g., cases before the Worker's Compensation Appeals Board (WCAB)). In particular, discovery can be shared that highlights a particular settlement position and most notably can be made a two-side ‘active argument’ that focuses settlement on those (few) meaningful issue(s) of difference. Hand-in-hand with the shared discovery (with mark-up or otherwise) and (especially) the few issues subject to ‘active argument’—a complete package of (required) settlement documentation can be prepared, packaged, and (upon acceptance by all (both) parties to a case filed and noticed.

In some implementations, the resolution of a qualified case can be effectively reduced to the (few) real disputes impactful to decision making and information focused thereon—ultimately speeding the resolution of a case. Further, as resolution is agreed, the agreement thereon can be submitted in real-time via a live-filing capability—preventing any retrenchment or revision thereon. And ultimately, the workflow automatically closes out the case, providing all of the proper (and formal) notices required under the relevant authority.

In some implementations, document management process 10 may allow parties to file a compromise and release. For example, an applicant party may be permitted to create and file a Compromise and Release Filing Packet with the relevant authority (e.g., California Division of Workers' Compensation Electronic Adjudication Management System (EAMS)). Document management process 10 may automatically complete the Compromise and Release Filing form using the case information provided by EAMS. The filing user may be permitted to edit the Compromise and Release Filing form in order to correct errors or to add additional information and may be permitted to add or remove case records and private documents to the filing packet. Document management process 10 may automatically create document cover sheets for the documents included in the filing packet. Document management process 10 may automatically create proof of service documents for the parties specified in the filing. Document management process 10 may file the Compromise and Release filing packet with the relevant authority (e.g., California Division of Workers' Compensation Electronic Adjudication Management System (EAMS)). Document management process 10 may capture the Compromise and Release filing packet batch ID during filing and may make it available in the case portal's filings case view. Document management process 10 may make the filed Compromise and Release filing packet available to all case parties in the case portal's filings case view. Document management process 10 may notify all case parties who are users of the case portal. Notices are reported to users via dashboards and made available to them through the case portal's filings case view.

In some implementations, document management process 10 may allow parties to file a stipulations with request for awards. For example, an applicant party may be permitted to create and file a Stipulations With Request for Awards filing packet with a relevant authority (e.g., the California Division of Workers' Compensation Electronic Adjudication Management System (EAMS)). Document management process 10 may automatically complete the Stipulations With Request for Awards filing form using the case information provided by EAMS. In some implementations, the filing user may be permitted to edit the Stipulations With Request for Awards filing form in order to correct errors or to add additional information. The Filing user may be permitted to add or remove case records and private documents to the filing packet. Document management process 10 may automatically create document cover sheets for the documents included in the filing packet. Document management process 10 may automatically create proof of service documents for the parties specified in the filing. Document management process 10 may file the Stipulations With Request for Awards filing packet with the relevant authority (e.g., California Division of Workers' Compensation Electronic Adjudication Management System (EAMS)). Document management process 10 may capture the Stipulations With Request for Awards filing packet batch ID during filing and may make it available in the case portal's filings case view. Document management process 10 may make the filed Stipulations With Request for Awards filing packet available to all case parties in the case portal's filings case view. Document management process 10 may notify all case parties who are users of the case portal. Notices may be reported to users via a dashboard and may be made available to them through the case portal's filings case view.

Referring also to the example of FIG. 25 and in some implementations, document management process 10 may provide a filings view. For example, the filings view may display a summary of all known case filings in tabular format. The Filings View may display case filing packet documents filed through Document management process 10. Notices to case parties may be visible in the filings case view. Notices for a particular case party may not be visible to other case parties in the filings case view. Users may be permitted to view and download documents contained in the case filings view.

Referring also to the example of FIG. 26 and as discussed above, document management process 10 may allow parties to file a compromise and release. For example, a user may select the My Filings view from the case views dropdown list. The user may select the Create New Filing control and may select File Compromise and Release from the filing types popup list control. Document management process 10 may display the File Compromise and Release form. The user may select the Select QME Reports button. Document management process 10 may display the Select QME reports form containing a list of all case documents. The user may select the appropriate QME reports from the list of case documents. Document management process 10 may return to the File Compromise and Release form. The user may enter the appropriate information in the Compromise and Release form. Fields that are marked with a red asterisk must be completed. The user may select a Generate Documents button. Document management process 10 may generate the required documents and assembles the filing packet including the document cover sheet, document separator sheets, compromise and release form, QME reports, and proof of service. Document management process 10 may display the filing packet on the user's screen. The user may review the filing packet information and then clicks on the submit filing button to file the Compromise and Release. Document management process 10 may submit the filing electronically and may add the Compromise and Release filing packet to the list of case documents. Document management process 10 may notify the other case parties which use Document management process 10 of the filing. Case parties which do not use Document management process 10 may be notified via mail.

Referring also to the example of FIG. 27 and in some implementations, allow parties to file a stipulations with request for awards. For example, a user may select the My Filings view from the case views dropdown list. The user may select the Create New Filing control and may select File Stipulation with Request for Awards from the filing types popup list control. Document management process 10 may display the File Stipulation with Request for Awards form. The user may select the Select QME Reports button and document management process 10 may display the Select QME reports form containing a list of all case documents. The user may select the appropriate QME reports from the list of case documents. Document management process 10 may return to the File Stipulations with Request for Awards form. The user may enter the appropriate information in the Stipulation with Request for Awards form. Fields that are marked with a red asterisk must be completed. The user may select the Generate Documents button. Document management process 10 may generate the required documents and may assemble the filing packet including the document cover sheet, document separator sheets, stipulation with request for awards form, QME report, and proof of service. Document management process 10 may display the filing packet on the user's screen. The user may review the filing packet information and then clicks on the submit filing button to file the Stipulation with Request for Awards. Document management process 10 may submit the filing electronically and may add the Stipulation with Request for Awards filing packet to the list of case documents. Document management process 10 may notify the other case parties which use document management process 10 of the filing. Case parties which do not use document management process 10 may be notified via mail.

### Active Authenticity

In some implementations, document management process 10 may apply unique technology-driven capabilities to control, protect, secure, and understand discovery/evidence. In particular, document management process 10 may expose the discovery/evidence into and through a blockchain that digitizes and secures the various steps of manual production (and digital transformation of the discovery/evidence), including the required chronological certification(s) required to maintain chain-of-custody certification; by immutable recording blocks of data amongst an individually undiscoverable distributed ledger for the evidence itself, but also by using Digital Rights Management (DRM) to token and record every change, transfer, and distribution along the way; together deployed and operated within a secure and independently certified information technology framework (typically referred to as HITRUST certified). In this way—the chain of data in the blockchain (and the inability for anything within the blockchain to be impermissibly changes) makes the authenticity of the evidence evident with the evidence itself.

With document management process 10, discovered information (evidence) may be automatically and effectively controlled, protected, and secured—ultimately facilitating the reliability of the evidence therein, in turn improving the accuracy of medical-legal evaluations required to proceed with treatment—or to onward confirm or deny such treatment in/for subsequent medical-legal evaluations. Additionally, by controlling, protecting, and securing the evidence, by default within a document management platform, less resource and time is required to manage and ‘prove’ the evidence—leaving more time and resource to focus on the information within the evidence itself. Finally, with medical information so well secured—the sharing of the medical facts and information therein, as necessary to inform treatment, can confidently be shared quickly and meaningfully to speed treatment decision making.

In some implementations, the source of a case record may be authenticated through a digital signature that ensures the authenticity of the document. The digital signature ensures that the authenticity of the case record has been verified by the entity whose digital signature was used to sign the document. Changes to the case record are prevented using normal signed Portable Document Format (PDF) mechanisms. A hash of the case record document may be generated when it is uploaded into a record management system (e.g., Med-Legal's record management system). This hash may be recorded in the initial block of the case record's blockchain along with the public key of the digital signature that was used to sign the document. The original hash of the case record may be validated whenever activity is performed against it to validate the authenticity of the record. The system may notify the user and any applications or services (e.g., Med-Legal) whenever it appears that the document has been tampered with. In some implementations, whenever an activity is performed against the case record (e.g., purchasing, viewing, downloading, sharing, etc.), a new block may be appended to the chain that contains the date and time that the activity was performed and the user who performed the activity. A hash for the block may be calculated based upon the hash of the previous block and the block's data and signed with a third party's (e.g., Med-Legal's Trust Certificate's) private key.

In some implementations, document management process 10 may use a proprietary, centralized ledger (e.g., a Trust Ledger) of the blocks in the chain of trust (e.g., blockchain). The Trust Ledger may be stored in a private data store. Users may be given the ability to view the source of the case record, its authenticity, and the history of actions that have been performed related to it.

In some implementations, case records may optionally include Digital Rights Management (DRM) features provided by a third-party platform. Case record DRM may prevent users from copying a case record document.

As discussed above, document management process 10 may be an internet based electronic system for parties in a case to conduct discovery related to and collaborate with others on those cases. Document management process 10 may provide a case portal (i.e., a collection of user interfaces that allow a user purchase, view, download, and share the case records that they are authorized to access). Document management process 10 may ensure the authenticity of the case records stored in its record repository.

In some implementations, document management process 10 may interact with a record custodian. A Record Custodian may include an organization who has the responsibility to manage and authorize the access to documents that are within its sphere of responsibility (case records). A Record Custodian may optionally provide another service (e.g., Med-Legal) with its digital certificate's public key. When it does so, the other service (e.g., Med-Legal) may validate that signed case records that have been provided by the Record Custodian has been signed with the appropriate digital certificate.

In some implementations, document management process 10 may interact with a Trust Ledger Data Store. For example, other services (e.g., Med-Legal) may maintain a chain of trust information for case records in its Trust Ledger Data Store. As authenticity blocks are created when actions are performed against case records and private documents, they are stored in the Trust Ledger. The Trust Ledger may include the information necessary to assure the authenticity and integrity of case records and private documents that are managed by document management process 10 along with their history.

Referring also to the example of FIG. 28 and in some implementations, document management process 10 may upload records. For example, the custodian may open an Internet Browser to a custodian records upload portal (e.g., the Med-Legal Secure Records Upload portal). The custodian may enter the information provided to them by another service (e.g., Med-Legal) into a Secure Record Upload portal and may click on the Enter button. Document management process 10 may redirect the custodian to a record upload page. The custodian may select the file(s) to be uploaded. The files may be added to a queue of files to upload. Document management process 10 may move to the beginning of the upload queue and determines if the upload queue is empty. If the upload queue is empty, document management process 10 may provide a final status for the upload queue to the user. If the upload queue is not empty, document management process 10 may retrieve the next file in the upload queue and may upload the file to a working folder on the server. Document management process 10 may determine whether the uploaded document has been digitally signed. If the document has been digitally signed, the digital signature may be validated against the custodian's digital signature in a data store (e.g., Med-Legal's data store). If the digital signature is not valid, the document may be removed from the working folder and an error message is returned to the user. If the document has not been digitally signed, the document may be digitally signed with a private key (e.g., Med-Legal's private key) and locked to prevent changes. The document may be moved to a record repository (e.g., the Med-Legal record repository). A new chain of trust may be created for the record. The initial block in the chain of trust may record the declared source of the document (e.g. the custodian), the owner of the digital signature used to sign the document, the date and time the document was uploaded, and the action performed (e.g. record uploaded). A new hash code may be calculated based upon the block's data and signed with a private key (e.g., Med-Legal's private key). The chain of trust block may be saved to the Trust Ledger Data Store. Document management process 10 may repeat until the upload queue is empty.

Referring also to the example of FIG. 29 and in some implementations, document management process 10 may import scanned records. For example, a user (e.g., a Med-Legal Field Representative) may scan the record's physical pages into a PDF file. The user (e.g., the Med-Legal Field Representative) may upload the scanned record to the record Quality Assurance (QA) queue. A reviewer (e.g., a Med-Legal reviewer) may perform quality control functions against the record. The validated document may be moved to a repository (e.g., the Med-Legal record repository). The document may be digitally signed with a private key (e.g., Med-Legal's private key) and locked to prevent changes. A new chain of trust may be created for the record. The initial block in the chain of trust may record the declared source of the document (e.g. the custodian), the owner of the digital signature used to sign the document, the date and time the document was uploaded, and the action performed (e.g. record scanned). A new hash code may be calculated based upon the block's data and signed with the private key (e.g., the Med-Legal's private key). The chain of trust block may be saved to the Trust Ledger Data Store.

Referring also to the example of FIG. 30 and in some implementations and as discussed above, document management process 10 may allow a user to upload private documents. In some implementations, the system may determine whether the uploaded document has been digitally signed. If the document has not been digitally signed, the document may be digitally signed with a private key (e.g., Med-Legal's private key) and locked to prevent changes. If the document has been digitally signed, a new chain of trust may be created for the record. The initial block in the chain of trust may record the declared source of the document (e.g. the customer), the owner of the digital signature used to sign the document, the date and time the document was uploaded, and the action performed (e.g. private document uploaded). A new hash code may be calculated based upon the block's data and signed with the private key (e.g., the Med-Legal's private key). The chain of trust block may be saved to the Trust Ledger Data Store.

Referring also to the example of FIG. 31 and in some implementations, a user may perform a recordable action against the document (e.g., purchase, view, download, share, etc.). The block may record the user account that was used to perform the action, the IP address of the computer that was used to perform the action, the location where the IP address is registered, the date and time the action was performed, and the action performed. A new hash code may be calculated based upon the preceding block's hash code and the block's data and may be signed with the private key (e.g., the Med-Legal's private key). The chain of trust block may be saved to the Trust Ledger Data Store.

In some implementations, document management process 10 may allow a user to review a record chain of trust. For example, a user may select the chain of trust icon associated with a record. Document management process 10 may redirect the user to the chain of trust page for the record or document. The chain of trust page may display a list of the blocks which make up the record/document's chain of trust in chronological order.

Referring also to the example implementation of FIG. 32, there is shown a diagrammatic view of client electronic device 38. While client electronic device 38 is shown in this figure, this is for example purposes only and is not intended to be a limitation of this disclosure, as other configurations are possible. Additionally, any computing device capable of executing, in whole or in part, document management process 10 may be substituted for client electronic device 38 (in whole or in part) within FIG. 8, examples of which may include but are not limited to computer 12 and/or one or more of client electronic devices 40, 42, 44.

In some implementations, client electronic device 38 may include a processor (e.g., microprocessor 3200) configured to, e.g., process data and execute the above-noted code/instruction sets and subroutines. Microprocessor 3200 may be coupled via a storage adaptor to the above-noted storage device(s) (e.g., storage device 30). An I/O controller (e.g., I/O controller 3202) may be configured to couple microprocessor 3200 with various devices (e.g., via wired or wireless connection), such as keyboard 3206, pointing/selecting device (e.g., touchpad, touchscreen, mouse 3208, etc.), USB ports, and printer ports. A display adaptor (e.g., display adaptor 3210) may be configured to couple display 3212 (e.g., touchscreen monitor(s), plasma, CRT, or LCD monitor(s), etc.) with microprocessor 3200, while network controller/adaptor 3214 (e.g., an Ethernet adaptor) may be configured to couple microprocessor 3200 to the above-noted network 14 (e.g., the Internet or a local area network).

The terminology used herein is for the purpose of describing particular implementations only and is not intended to be limiting of the disclosure. As used herein, the singular forms “a”, “an” and “the” are intended to include the plural forms as well, unless the context clearly indicates otherwise. As used herein, the language “at least one of A, B, and C” (and the like) should be interpreted as covering only A, only B, only C, or any combination of the three, unless the context clearly indicates otherwise. It will be further understood that the terms “comprises” and/or “comprising,” when used in this specification, specify the presence of stated features, integers, steps (not necessarily in a particular order), operations, elements, and/or components, but do not preclude the presence or addition of one or more other features, integers, steps (not necessarily in a particular order), operations, elements, components, and/or groups thereof.

The corresponding structures, materials, acts, and equivalents (e.g., of all means or step plus function elements) that may be in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications, variations, substitutions, and any combinations thereof will be apparent to those of ordinary skill in the art without departing from the scope and spirit of the disclosure. The implementation(s) were chosen and described in order to explain the principles of the disclosure and the practical application, and to enable others of ordinary skill in the art to understand the disclosure for various implementation(s) with various modifications and/or any combinations of implementation(s) as are suited to the particular use contemplated.

Having thus described the disclosure of the present application in detail and by reference to implementation(s) thereof, it will be apparent that modifications, variations, and any combinations of implementation(s) (including any modifications, variations, substitutions, and combinations thereof) are possible without departing from the scope of the disclosure defined in the appended claims.

What is claimed is:

1. A computer-implemented method comprising:
receiving, using a processor, information relating to a case being adjudicated at an electronic storage system;
identifying, using a categorization process, on or more evidentiary elements relevant to the case being adjudicated;
displaying information at a graphical user interface, the information including a view of a history, progression and a status of a medical condition associated with the case being adjudicated; and
generating a report or analysis based upon, at least in part, the information.

2. The computer-implemented method of claim 1, further comprising:
encoding case record information in a Record Annotation Markup Language “RAML” file

3. The computer-implemented method of claim 1, further comprising:
generating a case record excerpt summary including at least one of dates of service, service provider, summary of pertinent facts, or a list of page numbers.

4. The computer-implemented method of claim 1, further comprising:
graphically displaying a chronological view of the case including at least one of case facts, dates, item description, case record title or case record type.

5. The computer-implemented method of claim 1, further comprising:
generating a real-time updated view of all evidence associated with the case being adjudicated.

6. The computer-implemented method of claim 1, further comprising:
rating one or more records with respect to a static or a dynamic list of factors.

7. The computer-implemented method of claim 1, further comprising:
displaying, at a graphical user interface, a complete medical history portal including a plurality of graphical user interfaces configured to allow a user to search or order one or more records.

8. The computer-implemented method of claim 1, further comprising:
displaying, at a graphical user interface, a record availability dashboard.

9. The computer-implemented method of claim 1, further comprising:
selecting, via a graphical user interface, one or more documents to include in a bundle of documents within an electronic storage system;
defining, via the user interface, accessibility constraints for the one or more documents; and
electronically sharing at least one document of the bundle of documents with another user based upon, at least in part, the defined accessibility constraints.

10. The computer-implemented method of claim 9, further comprising:
automatically adding one or more case documents to the bundle of documents

11. The computer-implemented method of claim 9, further comprising:
creating, editing, or deleting a bundle of documents.

12. The computer-implemented method of claim 9, further comprising:
enabling a bundle updating service configured to identify one or more case records to update one or more live bundles with all available records.

13. The computer-implemented method of claim 1, further comprising:
generating a subset of disputes of the case being adjudicated.

14. The computer-implemented method of claim 13, further comprising:
generating one or more active arguments related to the subset of disputes.

15. The computer-implemented method of claim 1, further comprising:
automatically generating one or more packaged settlement documents

16. The computer-implemented method of claim 1, further comprising:
authenticating the information using one or more of blockchain, digital rights management, digital signatures, or encryption processes.

17. The computer-implemented method of claim 16, wherein the blockchain process includes a trust ledger data store.

18. The computer-implemented method of claim 1, further comprising:
displaying, at a graphical user interface, a current and verified capability to track, view, personally utilize, notate, add private documentation, share, or report on all evidentiary discovery elements that are active and in use for a case by a case party or authorized participant for an adjudicated case.

19. The computer-implemented method of claim 1, further comprising:
displaying, at a graphical user interface, a current, immediate, and verified view to active and authorized case parties for the case being adjudicated.

20. The computer-implemented method of claim 1, further comprising:
performing updating operations regarding one or more data updates to an Electronic Adjudication Management System “EAMS” using one or more synchronization engines.
